(19) United States
(12) Patent Application Publication
(10) Pub. No.: US 2011/0042241 A1
(43) Pub. Date: Feb. 24, 2011

(54) Electrochemical Assays

(75) Inventors: Angelo Kotsis, New South Wales (AU); John Brown, Mosman (AU); Claudia Bonifer, Maroubra Beach (AU); Peter McMillan, Wellington Downs (AU); John Parselle, Belleair, FL (US)

Correspondence Address: BANNER & WITCOFF, LTD., 1100 13th STREET, N.W., SUITE 1200, WASHINGTON, DC, 20005-4051, US

(73) Assignee: OXTOX LIMITED, London (GB)

(21) Appl. No.: 12/810,366

(22) PCT Filed: Dec. 22, 2008
(86) PCT No.: PCT/GB08/04246

§ 371 Date: Oct. 22, 2010

(30) Foreign Application Priority Data
Dec. 24, 2007 (GB) 0725234.9

(51) Int. Cl. G01N 33/487; G01N 27/26

(52) U.S. Cl. 205/792; 204/407; 204/403.02; 204/400; 204/435; 204/412

(57) ABSTRACT

A fluid assay system comprises a swab having an absorbent portion for receiving and retaining a fluid sample. The system also comprises an electrode module having at least one electrode, arranged so that it can be selectively placed over the absorbent portion of the swab in order to contact the electrode with the fluid sample. The system further comprises a reader module configured to process signals from the electrode module, with the electrode and reading modules arranged such that they can be coupled together for communication of the signals from the electrode module to the reader module.

This invention relates to electrochemical assays for determining the presence or quantity of analyte in a fluid sample, e.g. a human bodily fluid, and to equipment for performing such assays.

A wide variety of assays performed on samples of human bodily fluids are known in the art. Conventionally fluid samples would need to be taken by a physician or other medical personnel and sent away to a laboratory for performance of the appropriate assay and analysis of the results. More recently, however, there has been a growth in the availability of assays which can be carried out in real time close to the subject from which the sample is obtained, i.e. without the need to send samples away for laboratory analysis. A good example of this is the measuring of glucose levels in blood using handheld electrochemical assay devices which can be operated by a person suffering from diabetes in order to allow them to measure their instantaneous blood glucose level to assist in the management of their condition.

The applicant has also appreciated that there are many other useful assays which can be carried out on other body fluids.

When viewed from a first aspect the present invention provides a fluid assay system comprising a swab having an absorbent portion for receiving and retaining a fluid sample, an electrode module comprising at least one electrode, said module being arranged such that it can be selectively placed over said absorbent portion in order to contact said electrode with said fluid sample; the system further comprising a reader module configured to process signals from said electrode module, wherein said electrode module and said reading module are arranged such that they can be coupled together for communication of said signals from said electrode module to said reader module.

Thus it will be seen by those skilled in the art that in accordance with the invention a sample can be collected on a swab and analysed by electrodes in an electrode module in combination with a reader. Whilst it is envisaged that there may be circumstances where the whole apparatus is designed to be single-use or where it might be appropriate to re-use a particular set of electrodes, preferably the electrode module is removable from the reader module such that it can be cleaned, or more preferably, disposed of. This is clearly advantageous from the point of view of avoiding cross-contamination and also, more generally, from the ability to increase the level of hygiene associated with use of the device. Thus, in such an embodiment it is envisaged that the swab and electrode module will both be designed to be used only once and then discarded.

It will be appreciated therefore that the invention extends to the combination of a swab having an absorbent portion for receiving and retaining a sample of fluid, and an electrode module comprising a plurality of electrodes and which can be placed selectively over said absorbent portion in order to effect contact between said electrodes and said fluid sample.

Preferably the electrode module and swab are designed for single use.

In one set of preferred embodiments, the electrode module can be locked in place over the absorbent portion of the swab member. This has several advantages. Firstly, it can help to ensure that there is proper registration between the absorbent portion and the electrode module. Secondly, it prevents unauthorised re-use of a swab or electrode module, thereby avoiding the risk of cross-contamination.

Thirdly, it enables hygienic disposal of the swab and electrode module by preventing inadvertent access to the fluid on the absorbent portion of the swab and/or the electrode module.

Such an arrangement is considered to be novel and inventive in its own right and thus when viewed from a further aspect the invention provides a swab for use in a fluid assay system, said swab comprising an absorbent portion for receiving and retaining a fluid sample, said arrangement further comprising an electrode module having at least one electrode, said module being arranged such that it can be placed selectively in a position over said absorbent portion in order to bring said electrode into contact with said fluid sample, said electrode module being lockable in said position in order to prevent re-use of the swab member.

In accordance with all aspects of the invention, there are many different ways in which the swab and electrode module can be configured in order to give the described functionality. For example, there are a variety of possible mechanisms for bringing the electrode module into position over the absorbent portion of the swab.

In one set of possible embodiments, the electrode module is, or is able to be, rotatably fitted to the swab so as to have an open configuration allowing access to the absorbent portion in order to collect a fluid sample; and a closed configuration in which the absorbent portion is inside the electrode module so that the electrodes thereof can come into contact with the fluid sample. The electrode module might be provided permanently on the swab or it might be provided separately and able to be manually clipped or otherwise attached to it.

In another set of embodiments, the electrode module can be slid over the absorbent portion of the swab member. Again, the electrode module could be permanently fitted to the swab and thus able to slide along it or, as presently preferred, the electrode module might fit over the absorbent portion, e.g. in the manner of a cap or sleeve fitted to an end of the swab member.

It is envisaged that the electrode module will contain all of the electrodes needed to carry out a particular assay. Typically this would comprise a working electrode, a counter electrode and a reference electrode (although such a configuration is not considered essential). However in an alternative possibility one or more of the electrodes could be provided on the swab itself—e.g. in such a position that the electrode is placed in contact with the fluid sample when the sample is received.

In accordance with all of the aspects of the invention previously set out, an electrode module is placed over an absorbent portion of a swab so that one or more electrodes thereof are brought into contact with the fluid sample retained in the absorbent portion. In preferred embodiments of all such aspects of the invention, either or both of the electrode module and the swab is configured to apply a contact pressure between said electrodes and the fluid sample retained in the absorbent portion. The Applicant has found that this increases the reliability of the electrochemical assay being carried out.

There are of course many possible mechanisms for doing this. For example, one or both elements may comprise resilient means to provide the desired contact pressure. Alternatively, the pressure could be provided by the action of sliding, rotating or otherwise moving the electrode module over the absorbent portion. One illustrative example of this might be mutually acting cam means.

In other embodiments of the features set out above, the pressure could be applied independently of the action of bringing the electrode module and absorbent portion into registration. For example, the pressure could be applied completely manually, perhaps by means of a suitable button or lever. Alternatively, a latch or detent might be manually released in order to apply said pressure with a pre-tensioned resilient arrangement. In one set of particularly convenient embodiments, the aforementioned contact pressure is provided by the action of locking the electrode module to the swab. The advantages of this latter feature have been set out hereinabove. It is further envisaged that this action could be coupled in turn to the action of bringing the electrode module into registration with the absorbent portion of the swab. The convenience of such an embodiment is clear—namely that simply by placing the electrode module onto the appropriate part of the swab, the appropriate contact pressure can be automatically applied and the electrode module locked in such a position at the same time.

Although in the embodiments depicted herein the swab of the invention is embodied as an elongate member with the absorbent portion at one end, i.e. in a similar configuration to a traditional swab, the invention is not limited to such arrangements and no specific features or limitations should be inferred from the use of the term “swab”. For example, the swab might be designed to be fitted to another member, tool, device or machine prior to use or prior to bringing the absorbent portion thereof into registration with the electrode module. To take an example, arrangements are envisaged whereby a reusable tool or device has a disposable swab part fitted thereto.

Moreover the reference to an absorbent portion of the swab should not be understood as excluding the possibility of the entire swab being absorbent or of the same material. For example, the fluid sample might be received by just part or indeed all of an absorbent article comprising the swab. In other words, the absorbent portion could constitute the whole of the swab. One example of this might be an absorbent pad which is taken into the mouth of a user to absorb saliva and then placed into an apparatus such that it is brought into contact with the electrode of an electrode module.

The above notwithstanding, some of the embodiments of the invention comprise an elongate swab with the absorbent portion at one end and a handling portion at the other end. In preferred examples of such embodiments, the swab is provided with means disposed between the absorbent portion and the handling portion to inhibit fluid running down the swab from the absorbent portion to the handling portion. Such means could comprise a recess, but preferably comprises a protruding barrier.

When viewed from a further aspect the invention provides a swab for collecting a fluid sample comprising an elongate body having an absorbent portion for receiving said fluid sample at one end and having a handling portion at the other end, the swab further comprising means disposed between said absorbent portion and said handling portion for inhibiting fluid from running from said absorbent portion to said handling portion.

The invention may be used with a wide variety of different electrochemical assays on a number of different types of fluids. In a set of presently preferred embodiments, the system is adapted for use with human saliva samples, i.e. the swab is sterile, and thus the invention is considered to extend to the use of apparatus or systems in accordance with any of the aspects of the invention previously set out for the electrochemical assaying of human saliva samples. The configuration and composition of the electrodes in the electrode module will, of course, depend upon the electrochemical assays to be performed. In one set of preferred embodiments, the electrodes are adapted to detect one of more of: phenols, phenolic compounds and phenol derivatives such as tetrahydracannabinol as is described in greater detail in WO 2006/134386 in order to allow the system to be used for testing for the use of cannabis by the subject.

In aspects of the invention in which an electrode module can be coupled to a reader module in order to transfer signals from one to the other, it is envisaged that a wireless coupling could be provided e.g. with the signals encoded on a radio, infrared or ultrasonic transmission. More preferably however a wired connection is provided for reasons of cost effectiveness. The electrode module could comprise means for performing some processing or filtering of the signals from the electrodes. In preferred embodiments, however, the electrode module simply permits a direct connection to the electrodes, with any such filtering and processing being carried out in the reader module. This is clearly consistent with making the electrode module disposable and producible at a minimum cost and therefore disposable.

Preferably the coupling between the electrode module and the reader module comprises a simple plug-and-socket arrangement. In preferred embodiments the reader module is configured to indicate whether the electrode module has been properly connected to it. In some embodiments the reader module may be configured to identify or verify the electrode module. There might be several reasons for doing this. It could, for example, be used to ensure that the electrode module is from an authorised source, or that it is within an authorised shelf life. Another possibility which is given by having a separate reader module and electrode module is that different electrode modules could be used with a common reader module. In such arrangements the reader module might then be configured to determine automatically what type of electrode module had been connected to it and to perform the appropriate analysis as a consequence.

The electrode module could be provided with the minimum set of electrodes to perform an assay. As previously mentioned this typically consists of a working electrode, a counter electrode and reference electrode, although this is not essential. However in accordance with some embodiments, one or more additional working electrodes is provided. When viewed from a further aspect the invention provides an electrode module for an electrochemical assay, comprising a plurality of working electrodes on a common substrate.

In one set of embodiments the plurality of working electrodes are configured to detect two or more analytes. For example each working electrode could be configured to detect a different analyte. The applicant has appreciated that in the context of a real time, on-the-spot assay it is advantageous to be able to test for more than one analyte simultaneously, as opposed to having to carry out a series of tests, especially if each requires a fresh sample. One potentially useful application of this would be a roadside driver impairment test where various substances known to cause impairment of driving function could be tested for, such as cannabis, cocaine, heroin etc. In one possible embodiment, the electrode module is adapted to detect cannabis and amphetamines simultaneously. In other particular embodiments it could be adapted to detect any or all substances from the group comprising cannabis, amphetamines, cocaine, opiates and benzodiazepines. Of course the skilled person will appreciate that any number and combination of substances can be tested for.

It will be appreciated that such arrangements are novel and inventive in their own right and thus when viewed from a further aspect the invention provides an electrochemical assay system comprising a plurality of electrodes adapted to detect a plurality of analytes in a single fluid sample. This aspect of the invention also extends to a disposable electrode module comprising a plurality of electrodes adapted to detect a plurality of analytes in a single fluid sample.

In another set of embodiments a plurality of working electrodes, conveniently identical to one another, is provided for a single given analyte. It effectively allows multiple parallel assays to be carried out simultaneously on the same sample which can be exploited by the use of appropriate statistical techniques to give an accurate result. Also in some instances it can reduce the volume of fluid required to give a reliable measurement. These are both important factors in the applications envisaged for this technology. Of course an electrode module embodying this feature could still carry respective sets electrodes for a plurality of analytes; each set may comprise one or more working electrodes.

The working electrodes could each form part of an independent set of electrodes for performing an assay—e.g. each working electrode could have its own associated reference and counter electrodes. Preferably however two or more working electrodes share a common reference or counter electrode.

In one set of preferred embodiments a plurality of working electrodes is arranged around a common reference or counter electrode, e.g. in a circle. This provides a compact arrangement of the electrodes in the electrode module on which it is convenient, in preferred embodiments for a swab carrying the sample to be placed

The number of working electrodes may be selected as appropriate for the application. To give some non-limiting examples there may be between 2 and 30 working electrodes, or between 5 and 20 or between 8 and 16. In one specific example there are 12.

Preferably the working electrodes are each less than 2 mm wide, more preferably less than 1 mm wide. Small working electrodes allow a large number working electrodes to be fitted into a small area, either as part of an electrode array or an electrode module, while still remaining independent. This is advantageous because the swab does not therefore have to be unduly large to provide a sample across the whole area of the working electrodes which will be able to return an accurate measurement with only a small sample. It also enables a highly accurate measurement to be made as a result of the large number of working electrodes.

The working electrodes could be connected together on the electrode module so as to act effectively as a single distributed electrode. Preferably however the plurality of working electrodes have individual contacts to allow electrical connection to be made to them individually. This allows the working electrodes to be addressed individually which enhances the accuracy of the measurement by allowing multiple independent measurements be made, therefore decreasing the statistical error on the measurement.

Preferably a reader module adapted to be used in association with the embodiments of the electrode module described above is configured to address the working electrodes in parallel. Addressing the electrodes in parallel allows the measurements at the electrodes to be made simultaneously which increases the speed with which the measurement can be taken. This is clearly advantageous when an instantaneous measurement is desired.

Preferably the contacts for the electrodes are provided along one edge of the electrode module. Contacts passing to the edge of the electrode module allow an easy push-in connection to be made with a or the reader module.

Certain preferred embodiments of the invention will now be described, by way of example only, with reference to the accompanying drawings in which:

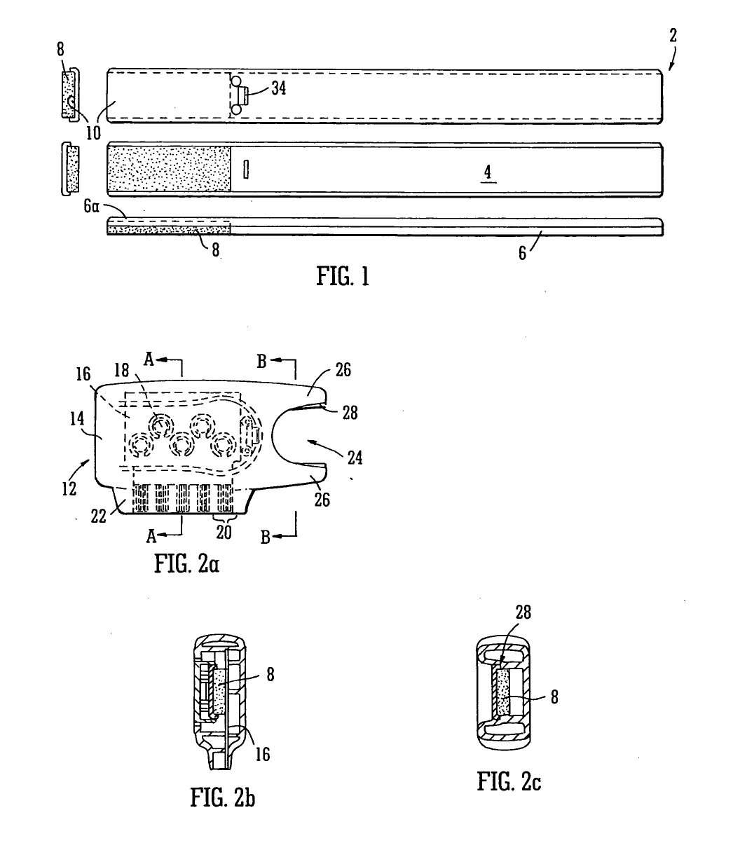

FIG. 1 shows various elevations of a swab in accordance with the invention;

FIG. 2a is a partially transparent view of an electrode module in accordance with the invention;

FIG. 2b is a cross-sectional view on line A-A of FIG. 2a;

FIG. 2c is a cross-sectional view on line B-B of FIG. 2a;

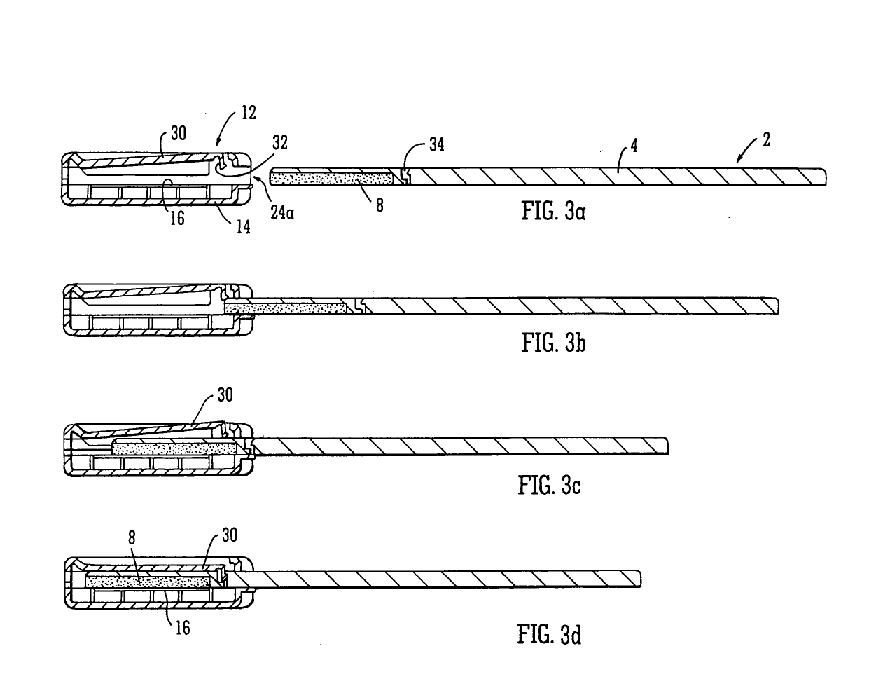

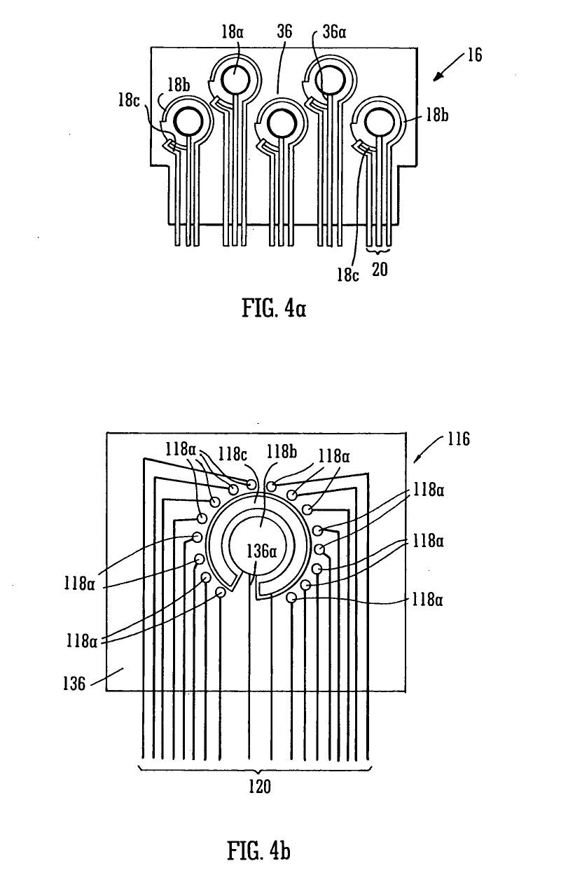

FIGS. 3a to 3d are respective cross-sectional views showing the swab of FIG. 1 being inserted into the electrode module;

FIG. 4a is a detailed view of an electrode assembly disposed within the electrode module;

FIG. 4b is a detailed view of the electrode assembly of another embodiment;

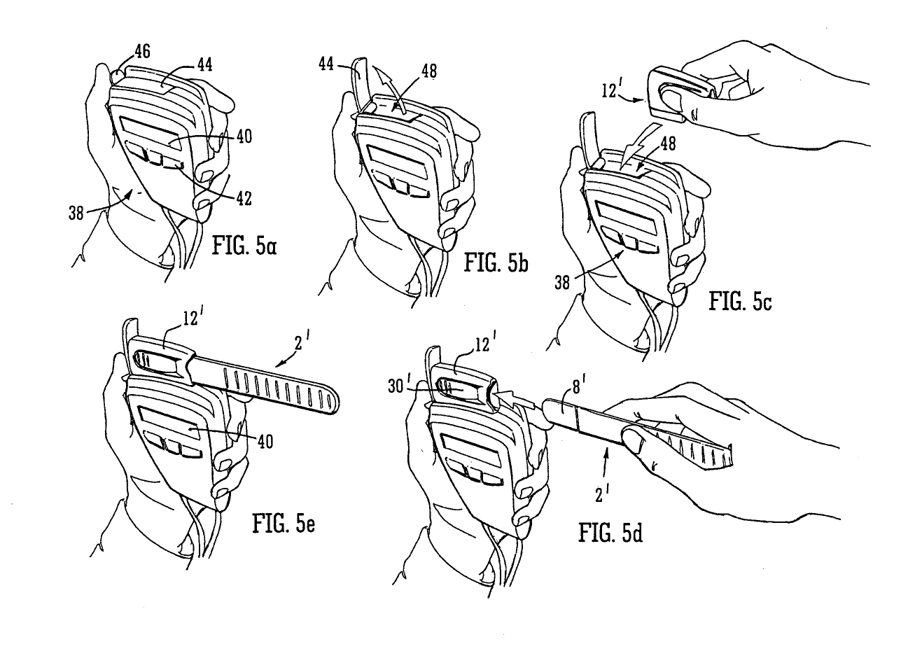

FIGS. 5a to 5e are respective diagrammatic representations showing the use of a swab and electrode module with a reader module according to a further embodiment of the invention;

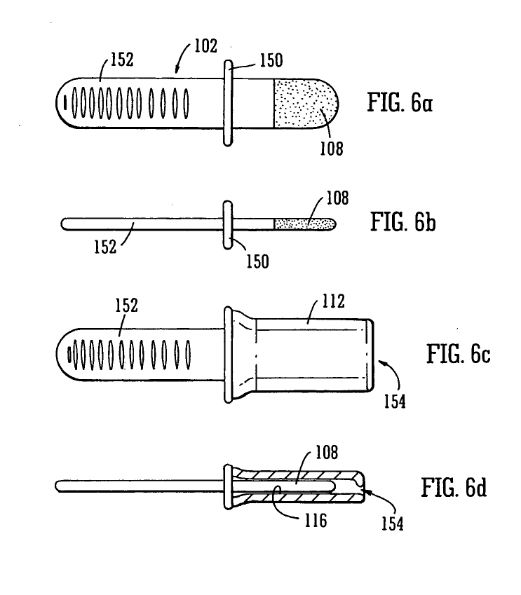

FIGS. 6 to 9 show various views of further alternative embodiments of the invention; and

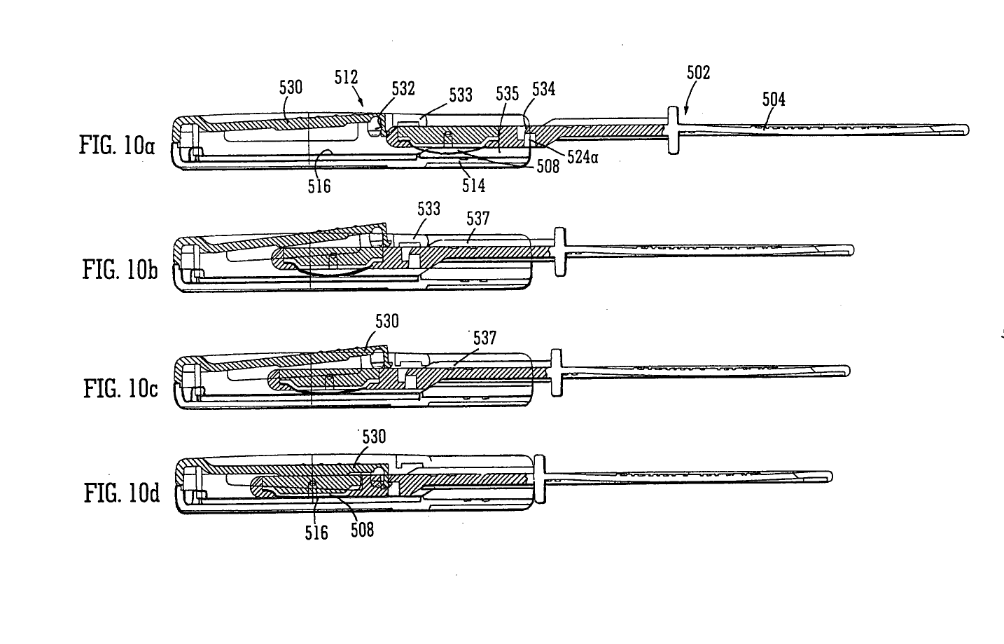

FIGS. 10a to 10d are respective cross-sectional views showing a swab being inserted into an electrode module.

FIG. 1 shows various elevations of a swab 2 in accordance with an embodiment of the invention. The swab comprises an elongate rectangular body section 4 which has two longitudinal vertically protruding walls along its respective outer edges. At the left-hand edge of the swab (as viewed from FIG. 1) there is a rectangular absorbent foam pad 8 attached to the upper face of the swab. A recess 10 is formed on the upper face of the body 4 in order to accommodate the absorbent pad 8. This can be seen from the end elevations to the left of FIG. 1. It will also be noted from the side elevation that the lateral walls of the swab 6a drop down adjacent the absorbent pad 8. Longitudinally behind the pad recess 10 is a recess 34, the purpose of which will become apparent later.

FIGS. 2a to 2c show the electrode module 12. In FIG. 2a the body 14 of the module is shown partly transparent in order to allow the inner structure thereof to be seen. Inside the main part of the module is an electrode assembly 16 comprising five independent electrode arrangements 18 (described in greater detail below with reference to FIG. 4). Each of the electrode arrangements 18 is connected to a respective set of three contact strips 20 which extend into a lateral extension of the electrode module body to form a plug portion 22.

At the right-hand end (as viewed from FIG. 2a) of the module 12 is a mouth portion 24 flanked by two lateral jaws 26 which define respective ledges 28 on their inner inwardly facing edges on which the edge of the swab 6a adjacent the absorbent pad 8 can slide (see FIG. 2c).

FIG. 2c is a section on line B-B showing the swab of FIG. 1 partially inserted into the electrode module 12. As the front end of the swab 2 is inserted into the mouth portion 24 of the module, the side walls 6a adjacent the absorbent pad 8 slide along the ledges 28 in order to keep the pad 8 clear of the edges of body 24a when the end of the swab is inserted into a longitudinal channel therein.

Referring now to FIG. 2b, it can be seen that when the swab is inserted further into the electrode module, the absorbent pad wipes over the electrode assembly 16. The pad 8 is pressed against the electrode module 16 by a hinged plate 30 which is described in more detail below with reference to FIGS. 3a to 3d.

FIGS. 3a to 3d show more clearly the stages in inserting the swab 2 into the electrode module 12. Thus initially, in this embodiment at least, the electrode module 12 is separate from the swab 2 and the swab 2 is inserted into the entrance 24a to the channel defined inside the module body 14. With reference to FIG. 3b, as the end of the swab 2 is inserted further into the electrode module 12 it slightly forces up a plate 30, hinged by a moulded hinge to the module body 14 defined, by means of a downwardly extending catch feature 32 at the distal edge thereof—in a cam-like manner.

As the swab 2 is inserted fully into the electrode module 12 (FIG. 3d) the catch feature 32 of the hinged plate 30 comes into alignment with a corresponding recess feature 34 defined in the body of the swab 4. This allows a user to squeeze the upper and lower faces of the electrode module 12 together which causes the hook feature 32 to pass into the corresponding recess 34 and lock in place against the undercut portion thereof. This maintains the contact pressure between the absorbent pad 8 and the electrode assembly 16 which has been found to give a more accurate and reliable measurement. Moreover, it also means that neither the electrode module 12 nor the swab 2 can be re-used, thereby avoiding the risk of cross-contamination and/or inaccurate results. It also means that when the assembly is discarded, no casual contact with the fluid sample retained in the absorbent pad 8 is possible, thereby allowing safe and hygienic disposal.

FIG. 4a is a close-up view of the electrode assembly 16 comprising five independent electrode sets 18. Each electrode set 18 comprises a working electrode 18a having a diameter of approximately 3 mm, a counter electrode 18b and a reference electrode 18c. These each have conductive tracks to take them to respective terminals 20 to allow electrical connection thereto in the reader. The working electrode 18a contains the appropriate chemical for detecting the substance of interest and varies between the five electrodes. For example it might include one of the chemicals disclosed in WO 2006/134386.

FIG. 4b is a close-up schematic view of an alternative electrode assembly 116 with a single electrode set 118 comprising sixteen working electrodes 118a which share a counter electrode 118b and a reference electrode 118c. In this embodiment the reference electrode 118c is shown as arcuate segment extending round the counter electrode 118b. However in alternative embodiments the reference electrode is of similar size to the working electrodes. Also in this embodiment the working electrodes are only of the order of 1 mm in diameter which allows all sixteen working electrodes 118a to be fitted compactly around the periphery of the reference and counter electrodes 118b, 118c.

The electrodes 118a, 118b, 118c each have individual conductive tracks to take them to respective terminals 120 to allow electrical connection with the reader. Since all the contacts are brought out to a common edge of the module, a convenient push-in connection between the electrode module and the reader can be used.

The working electrodes 118a all contain same chemical for detecting the substance of interest. Again this could be one of the chemicals disclosed in WO 2006/134386. As they have their own conductive tracks and terminals 120 the working electrodes 118a can be addressed individually and in parallel, allowing multiple simultaneous measurements to be made in use. This gives a very high measurement accuracy. A simple average of the measurements could be used, although more sophisticated techniques could be used, e.g. to exclude values much lower than the rest which might arise form a particular working electrode not being properly covered with the analyte fluid.

The electrode assemblies 16, 116 can be produced in the same way. Production begins with an electrically insulating substrate made of polypropylene, but any other suitable material could be used. On top of this is laid a layer of carbon, e.g. using screen printing as is well known in the art, which forms the basis of the three electrodes 18a, 118a; 18b, 118b; 18c, 118c of each electrode set 18, 118 and also the conductive tracks to the terminals 20, 120. A layer of silver chloride ink is then added to form the reference electrode 18c, 118c.

Thereafter an insulating dielectric layer 36, 136 is printed which covers most of the area of the assembly 16, 116 except for circle apertures over the area around the electrodes 18a, 118a; 18b, 118b; 18c, 118c although there is a tab 36a, 136a which extends along the conductive track for each reference 18c, 118c.

Finally the appropriate reagents are placed on the working electrodes 18a, 118a.

FIGS. 5a to 5e show various steps in the use of an assay system in accordance with an embodiment similar to those described above. FIG. 5a shows schematically a hand-held reader module 38 which has a visual display 40 on the front, operating buttons 42 below the display and a hinged flap 44 at the top which is openable by a small thumb lever 46 on the other side of the hinge.

When a user wishes to carry out an assay, the first step, shown in FIG. 5b, is to open the hinged upper flap 44 by depressing the thumb button 46. This reveals a socket 48 with a plurality of electrical connections inside (not visible) for connecting to the plug portion of the electrode module (see element 22 of FIG. 2a).

FIG. 5c shows the electrode module 12 being inserted into the socket 48 at the top of the reader module 38. Although not shown, in practice the electrode module 12′ would normally be stored in a sealed packet which might, if required, be sterile.

Once the reader module 12′ has been inserted into the reader module, the sample can be collected by taking a swab 2′, again from sealed and possibly sterile packaging, and a fluid sample, e.g. saliva/oral fluid, collected from a subject, onto the absorbent portion 8′ at one end. This absorbent portion 8′ is then inserted into the electrode module 12′ (see FIG. 5d) in a manner similar to that previously described in greater detail with reference to FIGS. 3a to 3d. Thus upon full insertion, the button 30′ is depressed to press the absorbent pad onto the electrode plate and to lock the swab 2′ into the electrode module 12′.

With the swab 2′ fully inserted into the electrode module 12′, the electrochemical assay can be carried out, with signals from the electrodes (not shown) being passed down to the reader module 38 through the plug-and-socket connection previously described. These signals can then be analysed and the results displayed on the display screen 40 in a manner known per se in the art. In the case of an electrode module having multiple working electrodes as shown for example in FIG. 4b, multiple measurements can be carried out simultaneously and the results combined statistically to produce a highly accurate aggregate result.

Once the assay has been satisfactorily completed, the electrode module 12′ can simply be removed from the socket in the top of the reader module 38, with the swab 2′ still locked inside it, and the two parts can then be safely and hygienically disposed of.

FIGS. 6a to 6d show an alternative embodiment of the invention. As will be seen, the swab 102 is somewhat similar in appearance to that previously described although it has a few differences. Firstly, the two ends of the swab are rounded and the absorbent pad 108 covers a larger proportion of the forward end. The other difference is that a protruding barrier 150 extends out of the plane of the swab all the way round and thus prevents any fluid which should happen to run out from the absorbent pad 108 and along the length of the swab from reaching the handle portion 152. This further enhances the hygiene associated with use of the swab.

The electrode module 112 visible in FIGS. 6c and 6d is broadly similar to that described in respect of the previous embodiments although here, electrical connection to the electrodes within the module is made by means of a socket 154 formed at the end of the module 112 opposite the end where the swab is inserted. Otherwise, operation of this embodiment is similar to that previously described and will not be repeated.

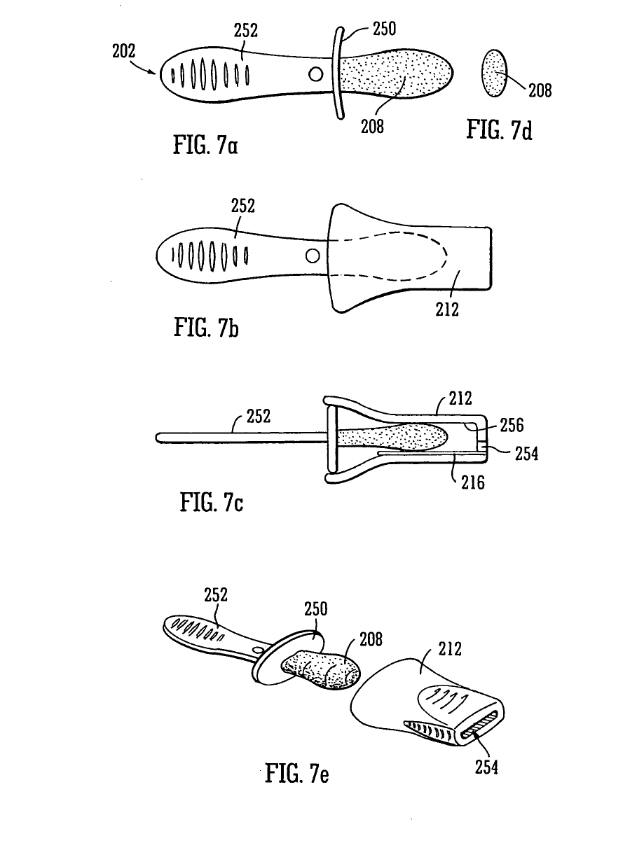

FIGS. 7a to 7e show a further embodiment of the invention. This is similar to the previously described embodiment although it will be seen that the swab 202 overall has a slightly different shape with the absorbent pad 208 filling the whole of the area of the swab forward of the barrier 250. It will also be seen from the end elevation of FIG. 7d that the absorbent portion 208 has an oval profile which might make it easier to use, particularly if a larger volume of fluid sample is required. The electrode module 212 is correspondingly slightly different in shape although has a very similar configuration to that of the previous embodiment. It will be seen from the section view of FIG. 7c that the profile of the absorbent portion 208 assists in providing a contact pressure onto the electrode assembly 216 as it is constrained by the width of the channel 256 inside the electrode module.

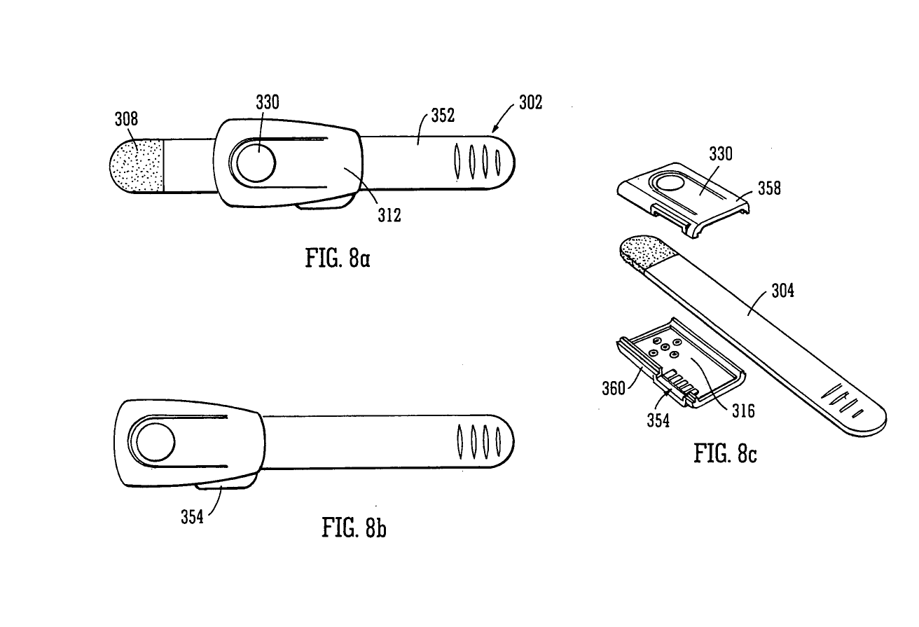

FIGS. 8a to 8c show a further embodiment of the invention. This embodiment is similar to that described with reference to FIGS. 6a to 6d except that in this embodiment the electrode module 312 is prior-fitted to the swab 302 such that it can slide along its length. Thus in the originally supplied configuration shown in FIG. 8a, the electrode module 312 is located part-way along the length of the swab 302, thereby exposing the absorbent portion 308 at the forward end. This allows a sample of fluid to be collected and indeed the presence of the electrode module 312 performs a similar function to the barriers 150, 250 shown in the embodiments of FIGS. 6 and 7. In another embodiment the electrode module could be configured to slide further back to a testing position to ensure that it does not hinder the taking of a sample before being moved over the absorbent pad for reading.

Once the sample has been collected, the electrode module can be slid along to the forward end of the swab so that the electrodes thereof are located over the absorbent portion 308. This is shown in FIG. 8b. As in the embodiment described with reference to FIGS. 1 to 3, the electrode module has a hinged plate 330 which is depressed to apply pressure to the absorbent pad 308 and to lock the module 312 in place.

FIG. 8c is an exploded view showing the construction of the electrode module. It comprises upper and lower shell sections 358, 360 which are snap-fitted together around the central part of the swab body 304. The upper shell section 358 has an arch-shaped slot defining the hinged plate 330. The lower section 360 carries the electrode assembly 316 which includes an electrical connection portion forming a socket 354 for connection to a corresponding reader (not shown).

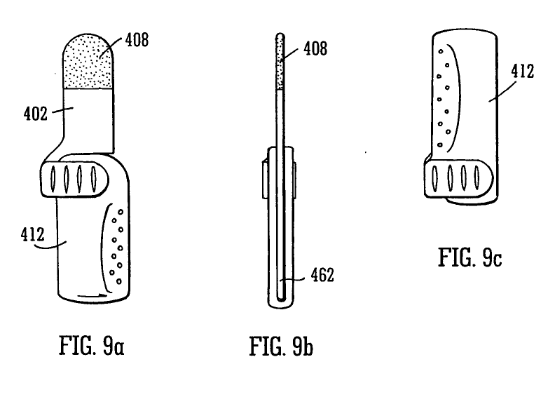

FIGS. 9a and 9b are respectively front and side elevations of the swab of a further embodiment in an open configuration; and FIG. 9c shows it in a closed configuration. In this embodiment the electrode module 412 is rotatably mounted on the swab 402 such that in the open configuration (FIGS. 9a and 9b) the electrode module 412 can act as a handle for the swab. When a sample has been collected on the absorbent portion 408, the swab section 402 can be rotated into an elongate slot 462 in the side of the electrode module 412, so that the swab section 402 is accommodated therein and the absorbent pad 408 is brought into contact with the electrodes inside it. The closed configuration is shown in FIG. 9c. A catch, detent or the like may be employed to prevent the swab section from being swung out again or it may simply become inaccessible when pushed fully home.

FIGS. 10a to 10d are similar to FIGS. 3a to 3d and show more clearly, for a different embodiment, the stages in inserting a swab 502 into an electrode module 512. Thus initially, in this embodiment at least, the electrode module 512 is separate from the swab 502 and the swab 502 is inserted into the entrance 524a to the channel defined inside the module body 514. With reference to FIGS. 10a and 10b, the end of the swab 502 is guided by a step 535 in the bottom moulding and a lip 533 in the top moulding of the module body 514 as it is inserted into the electrode module 512. As it is inserted further, the swab 502 slightly forces up a plate 530, hinged by a moulded hinge to the module body 514 defined, by means of a downwardly extending catch feature 532 at the distal edge thereof—in a cam-like manner.

The swab 502 is inserted in a straight line until the side ribs 537 of the swab 502 meet the lip 533 of the module body 514 as shown in FIG. 10b. In FIG. 10c it can be seen that the angled face of the side ribs 537 force the swab 502 to travel towards the bottom moulding of the module body 514. This action helps in generating a contact pressure between the absorbent pad 508 of the swab 502 and the electrode assembly 16.

As the swab 502 is inserted fully into the electrode module 512 (FIG. 10d) the catch feature 532 of the hinged plate 530 comes into alignment with a corresponding recess feature 534 defined in the body of the swab 504. This allows a user to press the hinged plate 530 which causes the hook feature 532 to pass into the corresponding recess 534 and lock in place against the undercut portion thereof. Alternatively the hinged plate 530 might be pressed by the action of inserting the electrode module 512 into the reader. This maintains the contact pressure between the absorbent pad 508 and the electrode assembly 516 which has been found to give a more accurate and reliable measurement. Moreover, it also means that neither the electrode module 512 nor the swab 502 can be re-used, thereby avoiding the risk of cross-contamination and/or inaccurate results. It also means that when the assembly is discarded, no casual contact with the fluid sample retained in the absorbent pad 508 is possible, thereby allowing safe and hygienic disposal.

It will be appreciated by those skilled in the art that only a small number of possible embodiments have been described and that many variations and modifications are possible within the scope of the invention. For example any of the features shown can be used with any other embodiment (whether or not described herein). Although testing of oral fluid for THC is used as an exemplary application of the principles of the invention this is not essential and there are many other possible assays that can be carried out either on oral fluid or on other types of fluid sample.

1-43. (canceled)

44. A fluid assay system comprising a swab having an absorbent portion for receiving and retaining a fluid sample, an electrode module comprising at least one electrode, said module being arranged such that it can be selectively placed over said absorbent portion in order to contact said electrode with said fluid sample; the system further comprising a reader module configured to process signals from said electrode module, wherein said electrode module and said reading module are arranged such that they can be coupled together for communication of said signals from said electrode module to said reader module.

45. A system as claimed in claim 44 wherein the electrode module is removable from the reader module.

46. A system as claimed in claim 44 wherein the electrode module and swab are designed for single use.

47. A system as claim in claim 44 wherein the electrode module can be locked in place over the absorbent portion of the swab member.

48. A system as claimed in claim 44 wherein the electrodes are adapted to detect one or more of: phenols, phenolic compounds and phenol derivatives.

49. A system as claimed in claim 44 wherein the electrode module comprises a direct connection to the electrodes, and the reader module comprises means for processing or filtering of the signals from the electrodes.

50. A system as claimed in claim 49 wherein the coupling between the electrode module and the reader module comprises a plug-and-socket arrangement.

51. A system as claimed in claim 50 wherein the reader module is configured to identify or verify the electrode module.

52. A system as claimed in claim 50 wherein the reader module is configured to determine automatically what type of electrode module is connected to it, and to perform an appropriate analysis.

53. A system as claimed in claim 44 wherein the electrode module is, or is able to be, rotatably fitted to the swab so as to have an open configuration allowing access to the absorbent portion in order to collect a fluid sample; and a closed configuration in which the absorbent portion is inside the electrode module so that the electrodes thereof can come into contact with the fluid sample.

54. A system as claimed in claim 44 wherein the electrode module can be slid over the absorbent portion of the swab member.

55. A system as claimed in claim 44 wherein the electrode module is permanently fitted to the swab.

56. A system as claimed in claim 44 wherein the electrode module can be fitted over the absorbent portion by a user.

57. A system as claimed in claim 44 wherein the electrode module comprises a working electrode, a counter electrode and a reference electrode.

58. A system as claimed in claim 44 wherein either or both of the electrode module and the swab is configured to apply a contact pressure between said electrodes and the fluid sample retained in the absorbent portion.

59. A system as claimed in claim 58 wherein either or both of the electrode module and the swab comprise resilient means to apply the contact pressure.

60. A system as claimed in claim 58 wherein the pressure is provided by the action of sliding, rotating or otherwise moving the electrode module over the absorbent portion.

61. A system as claimed in claim 58 comprising means for applying the pressure independently of the action of bringing the electrode module and absorbent portion into registration.

62. A system as claimed in claim 58 wherein the contact pressure is provided by the action of locking the electrode module to the swab.

63. A system as claimed in claim 62 wherein said locking action is coupled to the action of bringing the electrode module into registration with the absorbent portion of the swab.

64. A system as claimed in claim 44 wherein the swab comprises an elongate member with the absorbent portion at one end.

65. A system as claimed in claim 44 wherein the swab is sterile for collecting human saliva samples.

66. A system as claimed in claim 44 wherein the electrode module comprises a plurality of working electrodes.

67. A system as claimed in claim 66 wherein two or more working electrodes share a common reference or counter electrode.

68. A system as claimed in claim 66 wherein the plurality of working electrodes is arranged around a common reference or counter electrode.

69. A system as claimed in claim 66 wherein the electrode module comprises between 2 and 30 working electrodes, preferably between 5 and 20 working electrodes, more preferably between 8 and 16 working electrodes.

70. A system as claimed in claim 66 wherein the working electrodes are each less than 2 mm wide, preferably less than 1 mm wide.

71. A system as claimed in claim 66 wherein the plurality of working electrodes are connected together on the electrode module so as to act effectively as a single distributed electrode.

72. A system as claimed in claim 66 wherein the plurality of working electrodes have individual contacts to allow electrical connection to be made to them individually.

73. A system as claimed in claim 66 wherein the contacts for the electrodes are provided along one edge of the electrode module.

74. Use of a system as claimed in claim 44 for the electrochemical assaying of human saliva samples.

75. An electrode module for an electrochemical assay, comprising a plurality of working electrodes on a common substrate.

76. An electrode module as claimed in claim 75 comprising a plurality of working electrodes for a single given analyte.

77. An electrode module as claimed in claim 75 wherein the plurality of working electrodes are configured to detect two or more analytes.

78. An electrode module as claimed in claim 75 wherein the electrode module is adapted to detect any or all substances from the group comprising cannabis, amphetamines, cocaine, opiates and benzodiazepines.

79. An electrode module as claimed in claim 75 wherein two or more working electrodes share a common reference or counter electrode.

80. An electrode module as claimed in claim 75 wherein the plurality of working electrodes is arranged around a common reference or counter electrode.

81. An electrode module as claimed in claim 75 wherein the electrode module comprises between 2 and 30 working electrodes, preferably between 5 and 20 working electrodes, more preferably between 8 and 16 working electrodes.

82. An electrode module as claimed in claim 75 wherein the working electrodes are each less than 2 mm wide, preferably less than 1 mm wide.

83. An electrode module as claimed in claim 75 wherein the plurality of working electrodes are connected together on the electrode module so as to act effectively as a single distributed electrode.

84. An electrode module as claimed in claim 75 wherein the plurality of working electrodes have individual contacts to allow electrical connection to be made to them individually.

85. An electrode module as claimed in claim 75 wherein the contacts for the electrodes are provided along one edge of the electrode module.

86. Use of an electrode module as claimed in claim 75 for the electrochemical assaying of human saliva samples.

87. A reader module adapted to be used in association with the electrode module as claimed in claim 75.

88. A reader module as claimed in claim 87 wherein the reader module is configured to address the working electrodes in parallel.

89. Use of a reader module as claimed in claim 87 for the electrochemical assaying of human saliva samples.

90. An electrochemical assay system comprising a plurality of electrodes adapted to detect a plurality of analytes in a single fluid sample.

91. A system as claimed in claim 90 comprising a disposable electrode module.

92. Use of a system as claimed in claim 90 for the electrochemical assaying of human saliva samples.
